SEQUENCE LISTING (4 sequences)

SEQ ID NO: 1 - DNA, 30 bp, Artificial Sequence
  Oligonucleotide to act as a PCR primer for cloning of purine nucleoside phophrylase of E.coli K-12
gtgaattcacaaaaaggataaaacaatggc

SEQ ID NO: 2 - DNA, 29 bp, Artificial Sequence
  Oligonucleotide to act as a PCR primer for cloning of purine nucleoside phophrylase of E.coli K-12
tcgaagcttgcgaaacacaattactcttt

SEQ ID NO: 3 - DNA, 720 bp, Escherichia coli
atggctaccccacacattaatgcagaaatgggcgatttcgctgacgtagttttgatgccaggcgacccgctgcgtgcgaagtatattgctgaaactttccttgaagatgcccgtgaagtgaacaacgttcgcggtatgctgggcttcaccggtacttacaaaggccgcaaaatttccgtaatgggtcacggtatgggtatcccgtcctgctccatctacaccaaagaactgatcaccgatttcggcgtgaagaaaattatccgcgtgggttcctgtggcgcagttctgccgcacgtaaaactgcgcgacgtcgttatcggtatgggtgcctgcaccgattccaaagttaaccgcatccgttttaaagaccatgactttgccgctatcgctgacttcgacatggtgcgtaacgcagtagatgcagctaaagcactgggtattgatgctcgcgtgggtaacctgttctccgctgacctgttctactctccggacggcgaaatgttcgacgtgatggaaaaatacggcattctcggcgtggaaatggaagcggctggtatctacggcgtcgctgcagaatttggcgcgaaagccctgaccatctgcaccgtatctgaccacatccgcactcacgagcagaccactgccgctgagcgtcagactaccttcaacgacatgatcaaaatcgcactggaatccgttctgctgggcgataaagagtaa

SEQ ID NO: 4 - protein, 239 aa, Escherichia coli
MATPHINAEMGDFADVVLMPGDPLRAKYIAETFLEDAREVNNVRGMLGFTGTYKGRKISVMGHGMGIPSCSIYTKELITDFGVKKIIRVGSCGAVLPHVKLRDVVIGMGACTDSKVNRIRFKDHDFAAIADFDMVRNAVDAAKALGIDARVGNLFSADLFYSPDGEMFDVMEKYGILGVEMEAAGIYGVAAEFGAKALTICTVSDHIRTHEQTTAAERQTTFNDMIKIALESVLLGDKE

(19) United States
(12) Patent Application Publication
(10) Pub. No.: US 2005/0074857 A1
(43) Pub. Date: Apr. 7, 2005

(54) Method for producing a pyrimidine nucleoside compound and a new pyrimidine nucleoside compound

(75) Inventors: Tadashi Araki, Mobara-shi (JP); Hitoki Miyake, Mobara-shi (JP); Toshihiro Oikawa, Mobara-shi (JP)

Correspondence Address: BURNS DOANE SWECKER & MATHIS L L P, POST OFFICE BOX 1404, ALEXANDRIA, VA, 22313-1404, US

(73) Assignee: Mitsui Chemicals, Inc., Tokyo (JP)

(21) Appl. No.: 10/891,096

(22) Filed: Jul. 15, 2004

(30) Foreign Application Priority Data
Jul. 18, 2003 (JP) 2003-199175

(51) Int. Cl. C07H 19/04; C12P 19/30

(52) U.S. Cl. 435/89; 536/26.1

(57) ABSTRACT

A method for producing a pyrimidine nucleoside compound includes reacting a sugar phosphate and pyrimidine base derivative using an enzyme having cytosine nucleoside phosphorylase activity, the pyrimidine base derivative being represented by general formula (I): 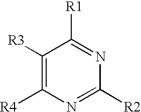 (wherein R1 represents an amino group that may be replaced with an acyl group having an alkyl group of 1 to 3 carbon atoms or an alkyl group of 1 to 3 carbon atoms, an alkyl group of 1 to 3 carbon atoms, or a thiol group; R2 represents an amino group, a thiol group, a hydroxyl group, or a hydrogen atom; R3 represents an alkyl group of 1 to 3 carbon atoms that may be replaced with a hydroxyl group, an amino group, or a hydrogen atom; R4 represents a hydroxyl group or a hydrogen atom; when R1 is an amino group, when R2 is a hydroxyl group, and when R4 is a hydrogen atom; R3 is neither an alkyl group of 1 to 3 carbon atoms nor a hydrogen atom).

### BACKGROUND OF THE INVENTION

1. Field of the Invention

The present invention relates to a method for producing a pyrimidine nucleoside compound useful as a synthetic material in, for example, medical products, and to a pyrimidine nucleoside compound.

2. Description of the Related Art

Japanese Unexamined Patent Application Publication Nos. 59-213397 and 5-49493 disclose methods for synthesizing a pyrimidine nucleoside compound using a pyrimidine base derivative under the presence of a nucleoside phosphorylase. According to the above methods, the nucleic acid base is a uracil base derivative, which has a carbonyl group disposed at the fourth position of the pyrimidine base. Methods for producing a nucleoside compound corresponding to the uracil base derivative using, for example, uracil, 5-halogenated uracil, or thymine are known.

In a method for producing a pyrimidine nucleoside compound using a sugar phosphate and a pyrimidine base derivative under the presence of a cytosine nucleoside phosphorylase, European Patent Publication No. 1254959A2 discloses a method for producing a cytosine nucleoside compound using 5-fluorocytosine, azacytosine, or 5-methylcytosine. However, no other methods are known.

In the production of a pyrimidine nucleoside compound, in particular, when the compound is used as a starting material of medical products, contamination due to a slight amount of by-products becomes a serious problem. In the production of a pyrimidine nucleoside compound by organic synthesis, in general, an a-isomer is generated as an isomer. The generation of the a-isomer increases the burden for purification and decreases the yield of the pyrimidine nucleoside compound. Unfortunately, these phenomena become a serious problem in industrial production.

### SUMMARY OF THE INVENTION

Accordingly, it is an object of the present invention to provide a method for synthesizing a pyrimidine nucleoside compound using a sugar phosphate and various pyrimidine base derivatives under the presence of an enzyme having cytosine nucleoside phosphorylase activity, and a pyrimidine nucleoside compound.

In order to solve the above problems, as a result of intensive study to produce a nucleoside compound using various pyrimidine base derivatives and a phosphorylated sugar under the presence of an enzyme having cytosine nucleoside phosphorylase activity, the present inventors have found that the use of a pyrimidine base compound represented by general formula (I), which will be described later, allows the production of a corresponding nucleoside compound.

Furthermore, the present inventors have studied a reaction for generating a pyrimidine nucleoside compound using 4-acetamidopyrimidine and pentose-l-phosphate under the presence of a microorganism having cytosine nucleoside phosphorylase activity. In the process, the present inventors have confirmed that a pyrimidine base or a pyrimidine nucleoside compound having a hydrolyzed acetyl group is generated in large quantity, thereby significantly decreasing the yield in the reaction.

The present inventors have found that this deacetylation is caused by the combination of the following reactions: (1) A reaction that chemically proceeds depending on the reaction conditions, and (2) A reaction due to an enzyme having deacetylation activity and generated by the host. Furthermore, as a result of intensive study of a method for suppressing the deacetylation, the present inventors have found the following methods are effective: In order to suppress the progress of the chemical deacetylation, the pH of the reaction mixture is controlled to be 6 to 9, preferably, 7 to 8, in addition, the reaction temperature is controlled to be 20° C. to 60° C., preferably, 30° C. to 40° C. In order to suppress the progress of the deacetylation due to the enzyme's deacetylation activity, the enzyme is decreased or removed from the bacterial cell of the microorganism, a culture solution of the bacterial cell, or a processed product thereof.

The present inventors have accomplished the present invention based on the above facts.

The present invention is as follows:

A method for producing a pyrimidine nucleoside compound includes the step of allowing a sugar phosphate to react with a pyrimidine base derivative under the presence of an enzyme having cytosine nucleoside phosphorylase activity, wherein the pyrimidine base derivative is represented by general formula (I): 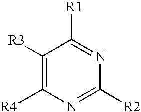
(wherein R1 represents an amino group that may be replaced with an acyl group having an alkyl group of 1 to 3 carbon atoms or an alkyl group of 1 to 3 carbon atoms, an alkyl group of 1 to 3 carbon atoms, or a thiol group; R2 represents an amino group, a thiol group, a hydroxyl group, or a hydrogen atom; R3 represents an alkyl group of 1 to 3 carbon atoms that may be replaced with a hydroxyl group, an amino group, or a hydrogen atom; R4 represents a hydroxyl group or a hydrogen atom; when R1 is an amino group, when R2 is a hydroxyl group, and when R4 is a hydrogen atom; R3 is neither an alkyl group of 1 to 3 carbon atoms nor a hydrogen atom);

The method for producing a pyrimidine nucleoside compound according to Item [1], wherein the pyrimidine base derivative represented by general formula (I) includes 2-hydroxy -4-methylpyrimidine, 4-acetamidopyrimidine, 2,4-diamino -6-hydroxypyrimidine, 4,5-diamino-6-hydroxypyrimidine, 2-thiocytosine, 5-hydroxymethylcytosine, or 4-thiouracil;

The method for producing a pyrimidine nucleoside compound according to Item [1] or Item [2], wherein the phosphorylated sugar includes ribose-l-phosphate, 2′-deoxyribose -1-phosphate, or 2′,3′-dideoxyribose-1-phosphate;

The method for producing a pyrimidine nucleoside compound according to any one of Items [1] to [3], wherein the enzyme is derived from E. coli;

The method for producing a pyrimidine nucleoside compound according to any one of Items [1] to [4], wherein the enzyme is provided from a bacterial cell of a microorganism having the enzyme, or a processed enzyme prepared from the bacterial cell or a culture solution of the bacterial cell;

A method for producing a pyrimidine nucleoside compound includes the step of allowing a compound represented by general formula (I), wherein R1 is an amino group replaced with an acyl group having an alkyl group of 1 to 3 carbon atoms, to react with a sugar phosphate using a bacterial cell of a microorganism or a processed enzyme of the bacterial cell of the microorganism having cytosine nucleoside phosphorylase activity, wherein deacylation activity of the microorganism to the acyl group in the compound is deactivated or reduced;

The method for producing a pyrimidine nucleoside compound according to Item [6], wherein the deacylation activity of the bacterial cell of the microorganism or the processed enzyme is deactivated or reduced by heating or placing in contact with water containing an organic solvent; and

A pyrimidine nucleoside compound represented by general formula (II): 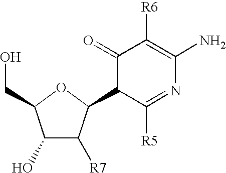
(wherein R5 represents an amino group or a hydrogen atom, R6 represents an amino group or a hydrogen atom, and R7 represents a hydroxyl group or a hydrogen atom).

A pyrimidine nucleoside compound has been synthesized only by organic synthesis so far. The present invention can provide a method for synthesizing a pyrimidine nucleoside compound under the presence of an enzyme.

### DESCRIPTION OF THE PREFERRED EMBODIMENTS

According to the present invention, an enzyme having cytosine nucleoside phosphorylase activity refers to an enzyme having activity for generating a cytosine nucleoside compound using cytosine or a cytosine derivative as a substrate. The enzyme may be derived from any origin such as animals, plants, and microorganisms as long as the above requirement is satisfied. Since an enzyme called a cytosine nucleoside phosphorylase is not generally known in the related field, the above term is defined as described above only for use in the present invention.

A preferable example of such a cytosine nucleoside phosphorylase includes an enzyme known as a purine nucleoside phosphorylase derived from a microorganism such as Escherichia coli of the genus Escherichia, the enzyme also having cytosine nucleoside phosphorylase activity. For example, sequence number 3 in the following sequence listing shows the DNA base sequence of the purine nucleoside phosphorylase derived from Escherichia coli and sequence number 4 in the sequence listing shows the amino acid sequence translated from the base sequence. In addition, because of the recent progress in genetic engineering, the amino acid sequence can be readily modified by deactivating, inserting, or replacing a part of the base sequence. In view of this level of technology, the cytosine nucleoside phosphorylase of the present invention also includes an enzyme wherein the amino acid sequence is modified by modifying a part of the base sequence as long as the desired enzyme activity is not affected, for example, as long as the enzyme activity can be maintained or improved. For example, in the amino acid sequence shown in sequence number 4, a few amino acids may be deleted, replaced, or added as long as the desired enzyme activity is not affected. The enzyme having this modified amino acid sequence may be used. In the base sequence shown in sequence number 3, the base sequence may be varied by, for example, deactivating, replacing, or adding amino acids as long as the desired enzyme activity is not affected and, in addition, the complementary sequence can be hybridized under stringent conditions. The enzyme may have the amino acid sequence coded by such a varied base sequence.

A pyrimidine base derivative according to the present invention is represented by general formula (I). In general formula (I), R1 represents an amino group that may be replaced with an acyl group having an alkyl group of 1 to 3 carbon atoms or an alkyl group of 1 to 3 carbon atoms, an alkyl group of 1 to 3 carbon atoms, or a thiol group; R2 represents an amino group, a thiol group, a hydroxyl group, or a hydrogen atom; R3 represents an alkyl group of 1 to 3 carbon atoms that may be replaced with a hydroxyl group, an amino group, or a hydrogen atom; and R4 represents a hydroxyl group or a hydrogen atom. In general formula (I), however, when R1 is an amino group, when R2 is a hydroxyl group, and when R4 is a hydrogen atom; R3 is neither an alkyl group of 1 to 3 carbon atoms nor a hydrogen atom.

It is generally known that pyrimidine bases form tautomers in an aqueous medium. For example, cytosine, 2-thiocytosine, and 6-hydroxy-4-aminopyrimidine, which are typical pyrimidine bases, form the following tautomers.

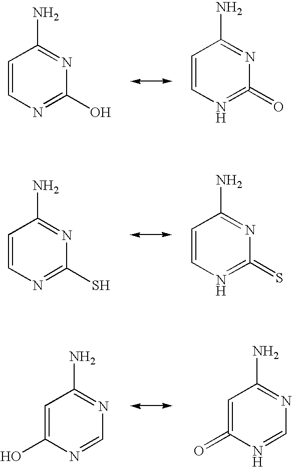

Accordingly, a pyrimidine base derivative represented by general formula (I) of the present invention also includes the corresponding tautomer.

Examples of the pyrimidine base derivative represented by general formula (I) include 2-hydroxy-4-methylpyrimidine, 4-acetamidopyrimidine, 2,4-diamino-6-hydroxypyrimidine, 4,5-diamino-6-hydroxypyrimidine, 2-thiocytosine, 5-hydroxymethylcytosine, and 4-thiouracil.

In a phosphorylated sugar according to the present invention, phosphoric acid is bonded at the first position of a polyhydroxyaldehyde, a polyhydroxyketone, or a derivative thereof by an ester bond. Preferable examples of the phosphorylated sugar include ribose-1-phosphate, 2′-deoxyribose-1-phosphate, 2′,3′-dideoxyribose-1-phosphate, and arabinose-1-phosphate.

Examples of the polyhydroxyaldehyde or the polyhydroxyketone derived from, for example, natural products include aldopentoses such as D-arabinose, L-arabinose, D-xylose, L-lyxose, and D-ribose; ketopentose such as D-xylulose, L-xylulose, and D-ribulose; and deoxy sugars such as D-2-deoxyribose and D-2,3-dideoxyribose. The polyhydroxyaldehyde or the polyhydroxyketone is not limited to the above.

These phosphorylated sugars are produced by, for example, phosphorolysis of a nucleoside compound using a nucleoside phosphorylase (J. Biol. Chem. Vol., 184, 437, 1950) or an anomer-selective chemical synthesis. In addition, the phosphorylated sugars are prepared by a method for synthesizing deoxyribose-1-phosphate under the presence of an enzyme described in PCT Publication No. WO01/14566.

According to the reaction for synthesizing a pyrimidine nucleoside compound of the present invention, a bacterial cell of a microorganism, a culture solution of the bacterial cell, or a processed product thereof is used, and appropriate reaction conditions such as the pH and the temperature are selected. The microorganism expresses a nucleoside phosphorylase that synthesizes a cytosine nucleoside compound using a cytosine derivative and a phosphorylated sugar as the substrates. The reaction is generally performed at the pH of 4 to 10, at the temperature of 10° C. to 80° C., and in an aqueous medium.

The aqueous medium includes a solvent composed of water or mainly composed of water, and having pH buffering capacity.

The concentration of the phosphorylated sugar and the pyrimidine base derivative used in the reaction is preferably about 0.1 to about 1,000 mM. The molar ratio of the pyrimidine base derivative to the phosphorylated sugar or the salt thereof is 0.1 to 10. In view of the conversion in the reaction, the molar ratio is preferably about 0.95.

Examples of the pyrimidine nucleoside compound generated in the present invention include 4-methylpyrimidine ribofuranosyl, 2′-deoxy-4-thiouridine, 2-thiocytidine, 5-hydroxymethylcytidine, N-acetyl-2′-deoxycytidine, and pyrimidine nucleoside compounds represented by general formula (II).

In the general formula (II), R5 represents an amino group or a hydrogen atom; R6 represents an amino group or a hydrogen atom; and R7 represents a hydroxyl group or a hydrogen atom.

According to the present invention, a bacterial cell of a microorganism, a culture solution of the bacterial cell, or a processed product thereof includes a product prepared by disrupting the bacterial cell wall of the microorganism or the bacterial cells in the culture solution with, for example, ultrasonic waves or osmotic shock, a product prepared by immobilizing the bacterial cells or the above disrupted cells with an immobilization support, and an enzyme purified from, for example, the disrupted cells.

In order to trap phosphoric acid generated in the reaction mixture, metal salts that are slightly soluble in phosphoric acid, or a support such as an ion-exchange resin may be added. Thus, the yield in the reaction can be increased.

A compound represented by general formula (I) wherein R1 is an amino group replaced with an acyl group having an alkyl group of 1 to 3 carbon atoms is allowed to react with a phosphorylated sugar under the presence of a bacterial cell of a microorganism, a culture solution of the bacterial cell, or a processed product thereof to generate the corresponding pyrimidine nucleoside compound. In the bacterial cell of the microorganism, the deacylation activity of an amino group replaced with an acyl group having an alkyl group of 1 to 3 carbon atoms is deactivated or decreased.

The deacylation activity of an amino group replaced with an acyl group having an alkyl group of 1 to 3 carbon atoms can be deactivated or decreased by heat treatment or a reducing treatment.

According to the present invention, the heat treatment is not limited as long as the deacylation activity is deactivated or decreased without deactivating the cytosine nucleoside phosphorylase. For example, a bacterial cell of the microorganism, a culture solution of the bacterial cell, or a processed product thereof is left to stand or is suspended in an aqueous medium for at least 10 minutes, preferably, at least 30 minutes. In this case, the pH of the mixture is generally 4.0 to 10.0, preferably, 6.0 to 9.0, and the temperature is generally at least 50° C., preferably, 60° C. to 80° C. The heating time is, more preferably, 40 hours or less.

The cytosine nucleoside phosphorylase is further stabilized by adding at least 1 mM, preferably 10 to 100 mM of a phosphorylated sugar to the solution.

According to the present invention, the treatment to reduce the deacylation activity is not limited as long as the deacylation activity of an acyl group having an alkyl group of 1 to 3 carbon atoms can be deactivated or decreased without deactivating the cytosine nucleoside phosphorylase. For example, a bacterial cell of the microorganism, a culture solution of the bacterial cell, or a processed product thereof is exposed to an organic solvent or is subjected to heat treatment. Alternatively, a solution of an enzyme prepared by disrupting the bacterial cell may be treated with, for example, an organic solvent or ammonium sulfate to precipitate the protein. The enzymes are fractionated, thereby removing only the enzyme having the deacylation activity.

According to the present invention, the organic solvent is not limited as long as the deacylation activity is deactivated. Examples of the organic solvent include polar solvents such as methyl alcohol, ethyl alcohol, propyl alcohol, butyl alcohol, dioxane, tetrahydrofuran, methyl ethyl ketone, and acetone; alcohols such as 1-hexanol, 2-methyl-1-pentanol, 4-methyl-2-pentanol, 2-ethyl-1-butanol, 1-heptanol, 2-heptanol, 3-heptanol, 1-octanol, 2-octanol, and 1-nonanol; esters such as propyl acetate, butyl acetate, isobutyl acetate, sec-butyl acetate, pentyl acetate, isopentyl acetate, cyclohexyl acetate, and benzyl acetate; hydrocarbons such as pentane, cyclopentane, hexane, 2-methylhexane, 2,2-dimethylbutane, 2,3-dimethylbutane, cyclohexane, methylcyclohexane, heptane, cycloheptane, octane, cyclooctane, isooctane, nonane, decane, dodecane, petroleum ether, petroleum benzine, ligroin, industrial gasoline, kerosene, benzene, toluene, xylenes, ethylbenzene, propylbenzene, cumene, mesitylene, and naphthalene; halogenated hydrocarbons such as dichloromethane, chloroform, carbon tetrachloride, dichloromethane, chlorobenzene, and dichlorobenzene; phenols such as cresol and xylenol; ketones such as methylisobutylketone and 2-hexanone; ethers such as dipropyl ether, diisopropyl ether, diphenyl ether, and dibenzyl ether; amides such as N,N-dimethylformamide, N,N-dimethylacetamide, N,N-dimethylbenzamide, N-methyl-2-pyrrolidone, N-methylformamide, N-ethylformamide, N-methylacetamide, formamide, acetylamide, and benzoic acid amide; ureas such as urea, N-N′-dimethylurea, tetramethylurea, and N,N-dimethylimidazolidinone; and sulfoxides such as dimethyl sulfoxide, diethyl sulfoxide, and diphenyl sulfoxide.

In particular, acetone is preferable among the above organic solvents.

According to the present invention, a treatment in an organic solvent for deactivating or decreasing the deacylation activity is not limited as long as the deacylation activity is deactivated or decreased without deactivating the cytosine nucleoside phosphorylase. For example, a bacterial cell of the microorganism, a culture solution of the bacterial cell, or a processed product thereof is left to stand or is suspended in an aqueous medium containing an organic solvent for, in general, 10 minutes to 40 hours, preferably, 1 hour to 20 hours. In this case, the pH of the mixture is generally 4.0 to 10.0, preferably, 6.0 to 9.0. The concentration of the organic solvent in water is, for example, at least 10 volume percent, preferably 20 at least volume percent, and more preferably, at least 30 volume percent. The temperature is generally at least 0° C., preferably, at least 20° C., and more preferably, from 50° C. to 80° C.

Alternatively, such an organic solvent may be added to the reaction mixture. This method also provides the same effect.

A compound wherein R1 in general formula (I) is an amino group replaced with an acyl group having an alkyl group of 1 to 3 carbon atoms is allowed to react with a phosphorylated sugar using a bacterial cell or a processed enzyme in which the deacylation activity is deactivated or decreased by the reducing treatment. As a result, a pyrimidine nucleoside compound is produced. Examples of the pyrimidine nucleoside compound include N-acetyl-2′-deoxycytidine.

In order to recover the pyrimidine nucleoside compound from the reaction mixture, the difference in the solubility in a solvent such as water between the derivative and the product can be utilized. Ion exchange or an adsorption resin can also be used for the same purpose.

### EXAMPLES

Although the present invention will now be described with reference to examples, the present invention is not limited to the following examples.

The identification of the resultant pyrimidine nucleoside compound was performed as follows: The reaction mixture was ultrafiltrated and was then purified with a silica gel column. The product was extracted and dried under a vacuum. The resultant product was analyzed by C¹³-NMR and H¹-NMR.

All resultant pyrimidine nucleoside compounds were quantitatively determined by high performance liquid chromatography. The conditions for the analysis are described below.

Column: Develosil ODS-MG-5, 4.6×250 mm (Nomura Chemical Co., Ltd.)

Column temperature: 40° C.

Flow rate of pump: 1.0 mL/min.

Detection wavelength: 254 nm in UV

Eluent: potassium primary phosphate (50 mM):methanol=8:1 (V/V)

### REFERENCE EXAMPLE 1

(Preparation of a Bacterial Cell that Produces Cytosine Nucleoside Phosphorylase)

E. coli chromosomal DNA was prepared as follows:

Escherichia coli K-12/XL-10 strain (Stratagene) was inoculated in an LB culture medium (50 mL) and cultivated overnight at 37° C. The bacterial cells were collected, and subjected to bacteriolysis with a lysing agent containing lysozyme (1 mg/mL). The lysate was treated with phenol. Subsequently, as with a common method, ethanol was added in order to precipitate the DNA. The precipitated DNA was recovered by spooling onto a glass rod and was washed before being subjected to polymerase chain reaction (PCR).

Oligonucleotides (synthesized by Hokkaido System Science Co., Ltd. on consignment) having base sequences shown in sequence numbers 1 and 2 in the following sequence listing were used as primers for the PCR. The oligonucleotides were designed based on the base sequence (GenBank accession No. AE000508 wherein the code area corresponds to the base numbers 11,531 to 12,250) of the deoD gene that codes a known purine nucleoside phosphorylase (hereinafter referred to as PNP) derived from E. coli. These primers have recognition sequences of restriction enzymes EcoRI and HindIII in the vicinity of the 5′ terminal and the 3′ terminal, respectively.

The PCR was performed using a PCR solution (0.1 mL) containing the above E. coli chromosomal DNA that is completely digested with the restriction enzyme HindIII (6 ng/μL), and the primers (3 μM each). In the PCR, one cycle included a denaturation performed at 96° C. for one minute, an annealing performed at 55° C. for one minute, and an extension performed at 74° C. for one minute. The cycle was repeated 30 times.

The above reaction product and a plasmid pUC18 (Takara Shuzo Co., Ltd.) were digested with EcoRI and HindIII, and were ligated with Ligation High (Toyobo Co., Ltd.) to form a recombinant plasmid. Escherichia coli DH5α was transformed with the recombinant plasmid. The transformant was cultivated in an LB agar medium containing ampicillin (Am) (50 μg/mL) and X-Gal (5-bromo-4-chloro-3-indolyl-β-D-galactoside), thereby preparing white colonies of a transformant having Am resistance.

A plasmid was extracted from the above transformant. The plasmid in which the desired DNA fragment was inserted was named pUC-PNP73. The base sequences of the DNA fragment that was introduced in the plasmid pUC-PNP73 was confirmed according to a general method for determining base sequences. Sequence number 3 in the following sequence listing shows the base sequence and sequence number 4 shows the amino acid sequence translated from the base sequence. It is known that the subunit of this enzyme has a molecular weight of about 26,000 and the enzyme expresses its activity as a hexamer. According to this enzyme, the optimal temperature is about 70° C and the optimal pH is from about 7.0 to about 7.5. The above transformant was named E. coli MT-10905.

E. coli MT-10905 strain was cultivated overnight while shaking at 37° C. in an LB culture medium (100 mL) containing Am (50 μg/mL). The culture solution was centrifuged at 13,000 rpm for 10 minutes. The resultant bacterial cells were suspended in 20 mL of a phosphate buffer (pH 7.5, 100 mM). The suspension was centrifuged again at 13,000 rpm for 10 minutes. The resultant bacterial cells were suspended in a solution (10 mL) of 2′-deoxyribose 1-phosphate di(monocyclohexylammonium) salt (50 mM, SIGMA).

### REFERENCE EXAMPLE 2

(Preparation of a Purified Cytosine Nucleoside Phosphorylase)

The bacterial cells in the suspension prepared in Reference Example 1 were disrupted with an ultrasonic disrupter. The resultant suspension was heated at 70° C. for 10 minutes, and was then centrifuged to prepare a solution of crude enzyme. The solution was added to a column (DEAE-Toyopearl by Tosoh Corporation, 3 cm×10 cm) that was equilibrated with a tris-HCl buffer (pH 7.5, 50 mM). The solution was eluted with a linear gradient of a NaCl solution from 50 mM to 500 mM to recover the active fraction. The eluent was recovered as a precipitate in a saturated solution of ammonium sulfate (70%). The precipitate was dialyzed in a phosphate buffer (pH 7.5, 10 mM). The dialyzed solution was added to a hydroxyapatite column (3 cm×15 cm) that was equilibrated with a phosphate buffer (pH 7.5, 10 mM). The solution was eluted with a linear gradient of a phosphate buffer (pH 7.5) from 10 mM to 50 mM to recover the active fraction. The enzyme solution was recovered as a precipitate in a saturated solution of ammonium sulfate (70%). The precipitate was dissolved in 1 mL of a phosphate buffer (pH 7.5, 10 mM). The solution was dialyzed in a tris-HCl buffer (pH 7.5, 10 mM) to prepare 2 mL of purified enzyme. The analytical result of this purified enzyme by sodium dodecyl sulfate (SDS)-polyacrylamide gel electrophoresis showed a single band.

### EXAMPLE 1

A reaction solution (1.0 mL) containing ribose 1-phosphate di(monocyclohexylammonium) salt (50 mM, SIGMA), 2-hydroxy-4-methylpyrimidine (10 mM, Tokyo Kasei Kogyo Co., Ltd.), a sodium acetate buffer (pH 8.0, 100 mM), and the suspension (0.1 mL) of the bacterial cells prepared in Reference Example 1 was heated at 50° C for one hour to perform the enzymatic reaction. The reaction solution was diluted for analysis. According to the analysis, a corresponding pyrimidine nucleoside compound was generated (6.9 mM).

### EXAMPLE 2

A reaction solution (1.0 mL) containing 2′-deoxyribose 1-phosphate di(monocyclohexylammonium) salt (50 mM, SIGMA), 4-acetamidopyrimidine (10 mM, ALDRICH), a potassium acetate buffer (pH 8.0, 100 mM), and the enzyme solution (0.1 mL) prepared in Reference Example 2 was heated at 50° C. for one hour to perform the enzymatic reaction. The reaction solution was diluted for analysis. According to the analysis, a corresponding pyrimidine nucleoside compound was generated (4.4 mM).

### EXAMPLE 3

Distilled water (15 g) was added to 2′-deoxyribose-1-phosphate diammonium salt (2.5 g) produced by a method described in Japanese Unexamined Patent Application Publication No. 2002-205996, 2,4-diamino-6-hydroxypyrimidine (0.63 g, ALDRICH), and magnesium carbonate (1.5 g) to prepare a reaction solution. The enzyme solution (5 mL) prepared in Reference Example 2 was added to the reaction solution and was kept at 30° C. for 20 hours to perform the enzymatic reaction.

The reaction solution was analyzed by liquid chromatography mass spectrometry (LC-MS) using an electrospray ionization (+) (ESI) method. Conditions for liquid chromatography

Column: Develosil ODS-UG5, 2.0×150 mm (Nomura Chemical Co., Ltd.)

Mobile phase: A: 0.1% (V/V) acetic acid/H₂O
B: 0.1% (V/V) acetic acid/CH₃CN
A/B=90/10

Flow rate: 1.0 mL/min.

Detection wavelength: 254 nm

The elution time of the product was 5.25 minutes, and the result by the mass spectrometry was as follows. Mass number (intensity peak): 127 (80), 243 (100), and 485 (20)

### EXAMPLE 4

Distilled water (15 g) was added to 2′-deoxyribose-1-phosphate diammonium salt (2.5 g) produced by a method described in Japanese Unexamined Patent Application Publication No. 2002-205996, 4,5-diamino-6-hydroxypyrimidine (1.5 g, LANCASTER), and magnesium carbonate (1.5 g) to prepare a reaction solution. The enzyme solution (5 mL) prepared in Reference Example 2 was added to the reaction solution and was kept at 30° C. for 20 hours to perform the enzymatic reaction.

The reaction solution was analyzed by LC-MS using the ESI (+) method as in Example 3.

The elution time of the product was 6.68 minutes, and the result by the mass spectrometry was as follows. Mass number (intensity peak): 127 (40), 243 (100), and 485 (10)

The precipitate was removed from the reaction solution by filtration, and the pH of the solution was then adjusted to 2.0 by adding hydrochloric acid. The solution was concentrated to about 5 g. A three-fold volume of isopropyl alcohol was added to one volume of the solution and the solution was crystallized. The crystals were filtered and were washed with the same amount of isopropyl alcohol. The crystals were dried under a vacuum, thus recovering the crystals of the product (about 0.6 g).

The results of ¹H NMR and ¹³C NMR were as follows: ¹H NMR (D₂O, 400 MHz, 20.1° C.):

δ8.29 (s, 1H), 6.31 (t, J=6.3 Hz, 1H), 4.48 (m, 1H), 4.12 (m, 1H), 3.87 (dd, J=3.4 & 12.5 Hz, 1H), 3.77 (dd, J=5.1 & 12.5 Hz, 1H), 2.54 (m, 1H), and 2.42 (m, 1H) ¹³C NMR (D₂O) δ (ppm):

160.38, 156.12, 147.22, 102.61, 89.80, 88.60, 72.99, 63.74, and 42.58

### EXAMPLE 5

A reaction solution (1.0 mL) containing 2′-deoxyribose 1-phosphate di(monocyclohexylammonium) salt (50 mM, SIGMA), 2-thiocytosine (10 mM, SIGMA), a sodium acetate buffer (pH 8.0, 100 mM), and the enzyme solution (0.1 mL) prepared in Reference Example 2 was heated at 50° C. for one hour to perform the enzymatic reaction. The reaction solution was diluted for analysis. According to the analysis, a corresponding nucleoside compound was generated (0.3 mM).

### EXAMPLE 6

A reaction solution (1.0 mL) containing 2′-deoxyribose 1-phosphate di(monocyclohexylammonium) salt (50 mM, SIGMA), 5-hydroxymethylcytosine (10 mM, SIGMA), a sodium acetate buffer (pH 8.0, 100 mM), and the enzyme solution (0.1 mL) prepared in Reference Example 2 was heated at 50° C. for one hour to perform the enzymatic reaction. The reaction solution was diluted for analysis. According to the analysis, a corresponding nucleoside compound was generated (0.1 mM).

### EXAMPLE 7

A reaction solution (1.0 mL) containing 2′-deoxyribose 1-phosphate di(monocyclohexylammonium) salt (50 mM, SIGMA), 4-thiouracil (10 mM, ALDRICH), a sodium acetate buffer (pH 8.0, 100 mM), and the enzyme solution (0.1 mL) prepared in Reference Example 2 was heated at 50° C. for one hour to perform the enzymatic reaction. The reaction solution was diluted for analysis. According to the analysis, a corresponding nucleoside compound was generated (4.2 mM).

### EXAMPLE 8

(Operation for Inhibiting Deacetylation Activity)

A suspension containing the bacterial cells prepared in Reference Example 1 was heated at 60° C. for 6 hours. The resultant suspension is referred to as a heat-treatment suspension of the bacterial cells.

Acetone was added to the suspension containing the bacterial cells prepared in Reference Example 1 such that the ratio became 70% V/V. The solution was stirred at 30° C. for one hour. The mixture was centrifuged to recover the bacterial cells. The bacterial cells were dried in air. The resultant bacterial cells are referred to as acetone-treatment bacterial cells.

The suspension containing the bacterial cells prepared in Reference Example 1 was disrupted with ultrasonic waves to prepare a mixture of crude enzyme. Acetone was added to the mixture of crude enzyme such that the ratio became 50% V/V. The mixture was centrifuged to remove the precipitate. Acetone was further added such that the ratio became 80% V/V. The mixture was centrifuged to recover the precipitate. The precipitate was dried. The resultant enzyme powder is referred to as an acetone powder of the bacterial cells.

### EXAMPLE 9

(Reaction by Enzyme wherein the Deacetylation Activity is Inhibited)

Distilled water (16.5 g) was added to 2′-deoxyribose 1-phosphate di(monocyclohexylammonium) salt (1.3 g, SIGMA) and 4-acetamidopyrimidine (0.6 g, ALDRICH). Magnesium acetate (1.3 g) was further added to the solution. (1) The above heat-treatment suspension of the bacterial cells (4 g), (2) the acetone-treatment bacterial cells (1.2 g), and (3) the acetone powder of the bacterial cells (0.4 g) were separately added to the solution. The three kinds of solution were kept at 30° C. for 6 hours to perform the enzymatic reaction. Sodium hydroxide or acetic acid was added to the solution such that the pH of the solution was adjusted to about 7.0 during the reaction. In Comparative Example, the suspension (4 g) containing the bacterial cells prepared in Reference Example 1 was added to the solution to perform the enzymatic reaction without controlling the pH.

Table 1 shows the results.

TABLE 1

| | Treatment | | Yield of | Yield of |
|---|---|---|---|---|
| | of | | corresponding | decomposition |
| | bacterial | pH | nucleoside | product |
| | cells | control | compound | (deoxycytidine) |
| Comparative | Untreated | Not | 55% | 30% |
| Example | | controlled | | |
| Example | Untreated | Controlled | 60% | 25% |
| Example | Heat- | | | |
| | treatment | Controlled | 80% | 10% |
| | Acetone- | | | |
| Example | treatment | Controlled | 80% | 15% |
| Example | Acetone | Controlled | 95% | 2% |
| | powder | | | |

1. A method for producing a pyrimidine nucleoside compound comprising the step of:
allowing a sugar phosphate to react with a pyrimidine base derivative under the presence of an enzyme having cytosine nucleoside phosphorylase activity, wherein the pyrimidine base derivative is represented by general formula (I): 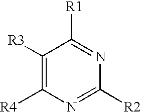
(wherein R1 represents an amino group that may be replaced with an acyl group having an alkyl group of 1 to 3 carbon atoms or an alkyl group of 1 to 3 carbon atoms, an alkyl group of 1 to 3 carbon atoms, or a thiol group; R2 represents an amino group, a thiol group, a hydroxyl group, or a hydrogen atom; R3 represents an alkyl group of 1 to 3 carbon atoms that may be replaced with a hydroxyl group, an amino group, or a hydrogen atom; R4 represents a hydroxyl group or a hydrogen atom; when R1 is an amino group, when R2 is a hydroxyl group, and when R4 is a hydrogen atom; R3 is neither an alkyl group of 1 to 3 carbon atoms nor a hydrogen atom).

2. The method for producing a pyrimidine nucleoside compound according to claim 1, wherein the pyrimidine base derivative represented by general formula (I) comprises 2-hydroxy-4-methylpyrimidine, 4-acetamidopyrimidine, 2,4-diamino-6-hydroxypyrimidine, 4,5-diamino-6-hydroxypyrimidine, 2-thiocytosine, 5-hydroxymethylcytosine, or 4-thiouracil.

3. The method for producing a pyrimidine nucleoside compound according to claim 2, wherein the phosphorylated sugar comprises ribose-1 -phosphate, 2′-deoxyribose-1-phosphate, or 2′,3′-dideoxyribose-1-phosphate.

4. The method for producing a pyrimidine nucleoside compound according to claim 3, wherein the enzyme having cytosine nucleoside phosphorylase activity is derived from E. coli.

5. The method for producing a pyrimidine nucleoside compound according to claim 4, wherein the enzyme having cytosine nucleoside phosphorylase activity is provided from a bacterial cell of a microorganism having the enzyme, or a processed enzyme prepared from the bacterial cell or a culture solution of the bacterial cell.

6. (canceled)

7. The method for producing a pyrimidine nucleoside compound according to claim 9, wherein the deacylation activity of the bacterial cell of the microorganism or the processed enzyme is selectively deactivated or reduced by heating or placing in contact with water containing an organic solvent.

8. A pyrimidine nucleoside compound represented by general formula (II): 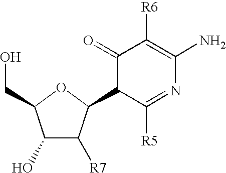
(wherein R5 represents an amino group or a hydrogen atom, R6 represents an amino group or a hydrogen atom, and R7 represents a hydroxyl group or a hydrogen atom).

9. A method for producing a pyrimidine nucleoside compound comprising the step of:
allowing a compound represented by general formula (I), 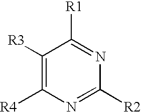
wherein R1 is an amino group replaced with an acyl group having an alkyl group of 1 to 3 carbon atoms, to react with a sugar phosphate using a bacterial cell of a microorganism or a processed enzyme of the bacterial cell of the microorganism having cytosine nucleoside phosphorylase activity, wherein deacylation activity of the microorganism to the acyl group in the compound is deactivated or reduced.

10. The method for producing a pyrimidine nucleoside compound according to claim 1, wherein the phosphorylated sugar comprises ribose-1-phosphate, 2′-deoxyribose-1-phosphate, or 2′,3′-dideoxyribose-1-phosphate.

11. The method for producing a pyrimidine nucleoside compound according to claim 10, wherein the enzyme having cytosine nucleoside phosphorylase activity is derived from E. coli.

12. The method for producing a pyrimidine nucleoside compound according to claim 2, wherein the enzyme having cytosine nucleoside phosphorylase activity is derived from E. coli.

13. The method for producing a pyrimidine nucleoside compound according to claim 1, wherein the enzyme having cytosine nucleoside phosphorylase activity is derived from E. coli.

14. The method for producing a pyrimidine nucleoside compound according to claim 13, wherein the enzyme having cytosine nucleoside phosphorylase activity is provided from a bacterial cell of a microorganism having the enzyme, or a processed enzyme prepared from the bacterial cell or a culture solution of the bacterial cell.

15. The method for producing a pyrimidine nucleoside compound according to claim 12, wherein the enzyme having cytosine nucleoside phosphorylase activity is provided from a bacterial cell of a microorganism having the enzyme, or a processed enzyme prepared from the bacterial cell or a culture solution of the bacterial cell.

16. The method for producing a pyrimidine nucleoside compound according to claim 11, wherein the enzyme having cytosine nucleoside phosphorylase activity is provided from a bacterial cell of a microorganism having the enzyme, or a processed enzyme prepared from the bacterial cell or a culture solution of the bacterial cell.

17. The method for producing a pyrimidine nucleoside compound according to claim 10, wherein the enzyme having cytosine nucleoside phosphorylase activity is provided from a bacterial cell of a microorganism having the enzyme, or a processed enzyme prepared from the bacterial cell or a culture solution of the bacterial cell.

18. The method for producing a pyrimidine nucleoside compound according to claim 3, wherein the enzyme having cytosine nucleoside phosphorylase activity is provided from a bacterial cell of a microorganism having the enzyme, or a processed enzyme prepared from the bacterial cell or a culture solution of the bacterial cell.

19. The method for producing a pyrimidine nucleoside compound according to claim 2, wherein the enzyme having cytosine nucleoside phosphorylase activity is provided from a bacterial cell of a microorganism having the enzyme, or a processed enzyme prepared from the bacterial cell or a culture solution of the bacterial cell.

20. The method for producing a pyrimidine nucleoside compound according to claim 1, wherein the enzyme having cytosine nucleoside phosphorylase activity is provided from a bacterial cell of a microorganism having the enzyme, or a processed enzyme prepared from the bacterial cell or a culture solution of the bacterial cell.
